(19) United States
(12) Patent Application Publication
(10) Pub. No.: US 2010/0114062 A1
(43) Pub. Date: May 6, 2010

(54) Methods and devices for providing oxygenated blood distal to an obstruction

(76) Inventors: Scott Wilson, Redwood City, CA (US); Kirk Pedersen, San Franciso, CA (US); Emily Vu, San Jose, CA (US)

Correspondence Address: HOEKENDIJK & LYNCH, LLP, P.O. BOX 4787, BURLINGAME, CA, 94011-4787, US

(21) Appl. No.: 12/589,865

(22) Filed: Oct. 29, 2009

Related U.S. Application Data
(63) Continuation of application No. 12/291,156, filed on Nov. 6, 2008.

(51) Int. Cl. A61M 25/14; A61M 25/00

(52) U.S. Cl. 604/508; 604/523; 604/527

(57) ABSTRACT

The catheter is positioned within an obstruction so that the distal end is positioned distal to the obstruction and proximal openings are positioned proximal to the obstruction. Blood enters the proximal openings and passes through the lumen to the distal end to perfuse blood distal to the obstruction.

### BACKGROUND OF THE INVENTION

The present invention is directed to methods and devices for providing oxygenated blood distal to an obstruction.

Obstructions in the vascular system cause damage to tissue distal to the obstruction since they reduce the supply of oxygenated blood to this tissue.

The present invention is directed to methods and devices for supplying oxygenated blood distal to an obstruction.

### SUMMARY OF THE INVENTION

The present invention provides a catheter, which is positioned within an obstruction to passively perfuse blood distal to the occlusion. The catheter has a lumen with openings in a proximal portion of the lumen that permits blood to enter the lumen.

The catheter is positioned within an obstruction so that the distal end is positioned distal to the obstruction and the proximal openings are positioned proximal to the obstruction. Blood enters the proximal openings and passes through the lumen to the distal end to perfuse blood distal to the obstruction.

These and other aspects of the present invention will become apparent from the following description of the preferred embodiments.

### DESCRIPTION OF DRAWINGS

FIG. 1 shows a catheter in accordance with the present invention.

FIG. 2 shows openings in the body of the catheter.

FIG. 3 shows the catheter positioned within an obstruction.

### DETAILED DESCRIPTION

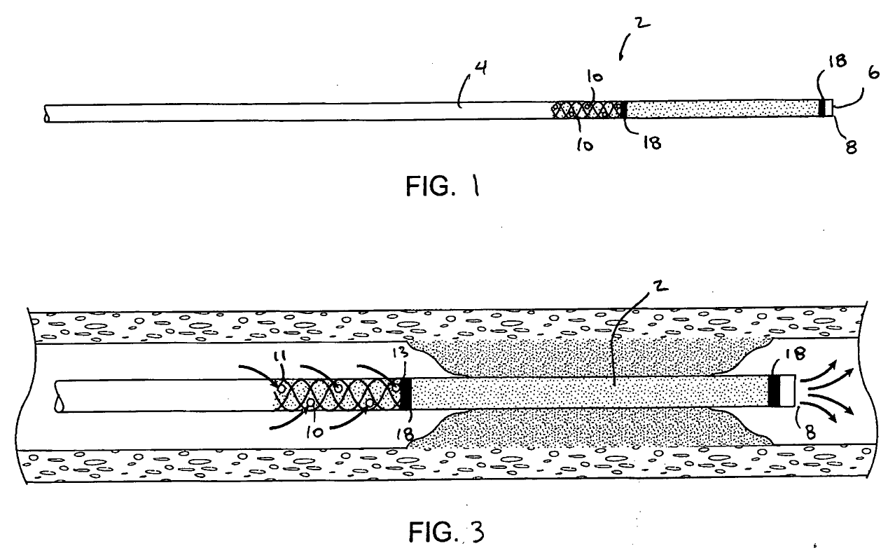

Referring to FIGS. 1-3, a catheter 2 in accordance with the present invention is shown. The catheter 2 has an elongate body 4 with a lumen 6 which terminates at a distal end 8. The catheter 2 has only one lumen 6 but may, of course, include other lumens without departing from various aspects of the present invention.

The catheter 2 has a plurality of openings 10 proximal to the distal end 8. As will be explained further below, the openings 10 permit blood to enter the catheter 2 so that the blood can pass through the lumen 6 to the distal end to perfuse blood distal to an obstruction. The catheter 2 may have 30-100 openings 10 which may be spaced apart and sized in the manner described below.

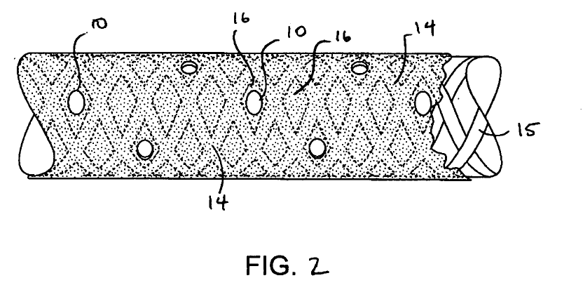

The openings 10 may take any suitable shape such as oval or round. Referring to FIG. 2, the openings 10 are shown as oval with a maximum dimension of 0.014 inch and a minor dimension of 0.009 inch. The area of the openings 10 may be 1.0-1.4 times the cross-sectional area of the lumen 6. The openings 10 may have a maximum dimension of 0.010-0.020 inch so that the openings 10 permit a sufficient flow of blood to pass therethrough while remaining relatively small enough that the structural integrity of the catheter 2 is not overly compromised.

The openings 10 extend along a length of 2-4 cm on the body 4 with the openings 10 in the preferred embodiment extending over a length of about 3 cm. The openings 10 may, of course, extend over a smaller or longer length of the body 4 without departing from various aspects of the invention. All of the openings 10 may be spaced 3-10 cm from the distal end 8 and the preferred embodiment has a proximal opening 11 spaced about 8 cm from the distal end 8 while a distal opening 13 is spaced about 5 cm from the distal end 8.

The body 4 has a reinforcing element 12 which may be a helical or braided reinforcement 14. FIG. 2 shows the braided reinforcement 14 with the openings 10 positioned in interstitial spaces 16 of the braided reinforcement 14. The reinforcing element 12 may be any suitable wire or ribbon and FIG. 2 shows a reinforcing ribbon 15 having a size of 0.0007″×0.003″ and made of stainless steel or nitinol. The body 4 may also have one or more radiopaque markers 18 near the distal end 8 as is known in the art.

Use of the catheter 2 is now described with reference to FIG. 3. The catheter 2 is advanced to an obstruction, which blocks a blood vessel. The catheter 2 is then advanced through the obstruction so that the distal end 8 is positioned distal to the obstruction and at least some of the openings 10, and preferably a substantial number of the openings 10, are positioned proximal to the obstruction. The catheter 2 may be advanced to the obstruction by passing the catheter 2 over another device, such as a guidewire or microcatheter, which is first advanced through the obstruction as is known in the art.

When the catheter 2 is positioned as shown in FIG. 3, blood enters the openings 10, passes distally through a short portion of the lumen 6, and exits the open distal end 8 to perfuse the tissue distal to the obstruction. The catheter 2 is left within the obstruction as a temporary therapy or may be the primary therapy if the obstruction naturally dissolves while the catheter 2 is in place. Alternatively, the obstruction may be removed by passing another device through the lumen 6 to remove, disrupt, dissolve or displace the obstruction. For example, an obstruction-removing element, such as those sold by Concentric Medical, may be used to remove the obstruction. Alternatively, a stent, angioplasty balloon or delivery of thrombolytics may be used to remove or displace the obstruction.

The present invention has been described in connection with a preferred embodiment; however, numerous modifications could be made without departing from the scope of the present invention. For example, the catheter may have a spiral reinforcing element, a tapered shape or additional openings proximal or distal to the plurality of openings.

1. A method of providing oxygenated blood distal to an obstruction, comprising the steps of:
providing a catheter having an elongate body and a lumen extending therethrough to a distal end of the body, the elongate body also having a plurality of openings in the body proximal of the distal end, the body having a braided reinforcement with the plurality of openings being positioned in interstitial spaces of the braided reinforcement, the body having 30-100 openings positioned 3-10 cm from the distal end;
positioning the catheter within an obstruction so that the distal end is distal to the obstruction and at least a portion of the plurality of openings are positioned proximal to the obstruction.

2-4. (canceled)

5. The method of claim 1, wherein:
the providing step is carried out with the openings having a maximum dimension of 0.010-0.020 inch.

6. A method of providing oxygenated blood distal to an obstruction, comprising the steps of:
providing a catheter having an elongate body and a lumen extending therethrough to a distal end of the body, the elongate body also having a plurality of openings in the body proximal of the distal end, the plurality of openings being positioned from 3-10 cm from the distal end, the openings having a maximum dimension of 0.010 to 0.020 inch;
positioning the catheter within an obstruction so that the distal end is distal to the obstruction and at least a portion of the plurality of openings are positioned proximal to the obstruction.

7. The method of claim 6, wherein:
the providing step is carried out with the body having a braided reinforcement, the plurality of openings being positioned in interstitial spaces of the braided reinforcement.

8. The method of claim 6, wherein:
the providing step is carried out with the catheter having 30-100 openings.

9. A catheter for perfusing blood distal to an obstruction, comprising:
an elongate body having a distal end;
a lumen extending through the elongate body to the distal end; and
a plurality of openings which extend through the body to the lumen.

10. The catheter of claim 9, wherein:
the body has a braided reinforcement, the plurality of openings being positioned in interstitial spaces of the braided reinforcement.

11. The catheter of claim 9, wherein:
the plurality of openings being positioned from 3-10 cm from the distal end.

12. The catheter of claim 9, wherein:
the plurality of openings number from 30-100 openings.

13. The catheter of claim 9, wherein:
the plurality of openings each have a maximum dimension of 0.010-0.020 inch.

14. A catheter for perfusing blood distal to an obstruction, comprising:
an elongate body having a distal end;
a lumen extending through the elongate body to the distal end;
a plurality of openings which extend through the body to the lumen;
the body having a braided reinforcement, the plurality of openings being positioned in interstitial spaces of the braided reinforcement.

15. The catheter of claim 14, wherein:
the plurality of openings being positioned from 3-10 cm from the distal end, each of the plurality of openings having a maximum dimension of 0.010 to 0.020 inch.

16. The catheter of claim 14, wherein:
the plurality of openings number from 30-100 openings.
